(19) United States
(12) Patent Application Publication
(10) Pub. No.: US 2012/0259400 A1
(43) Pub. Date: Oct. 11, 2012

(54) FLEXIBLE INTRALUMINAL SCAFFOLD

(75) Inventors: Randolf Von Oepen, Aptos, CA (US); Kevin J. Ehrenreich, San Francisco, CA (US)

(73) Assignee: Abbott Laboratories, Abbott Park, IL (US)

(21) Appl. No.: 13/350,529

(22) Filed: Jan. 13, 2012

Related U.S. Application Data
(60) Provisional application No. 61/433,055, filed on Jan. 14, 2011.

(51) Int. Cl. A61F 2/86; A61F 2/84

(52) U.S. Cl. 623/1.11; 623/1.1; 623/1.42; 623/1.34

(57) ABSTRACT

Expandable intraluminal scaffold defining a longitudinal axis is provided, wherein the scaffold includes at least two filaments extending from a head portion disposed along the longitudinal axis at a first longitudinal end, each of the at least two filaments including a free end portion at a second longitudinal end opposite the head portion. The at least two filaments converge toward each other at a juncture disposed proximate the longitudinal axis between the first longitudinal end and the second longitudinal end. A system including a delivery system and the intraluminal scaffold, as well as a method of delivering the scaffold, is also provided.

### CROSS REFERENCES TO RELATED APPLICATIONS

This application claims priority to U.S. Provisional Application No, 61/433,055, filed Jan. 14, 2011, the disclosure of which is hereby incorporated by reference in its entirety.

### FIELD OF DISCLOSED SUBJECT MATTER

The disclosed subject matter relates to an intraluminal scaffold for deployment in a body lumen of a patient, as well as the delivery method thereof. More particularly, the disclosed subject matter relates to an intraluminal scaffold having characteristics and properties suitable for being implanted in a vein.

### BACKGROUND

As well recognized, the cardiovascular or the circulatory system of human and other warm blood animals generally comprises the arterial system and the venous system. In its simplest form, the arterial system includes the blood vessels or arteries, through which blood travels from the heart, whereas the venous system includes the blood vessels or veins, through which blood travels to the heart. As contrasted by arteries, which are relatively thick-walled blood vessels, veins generally have thinner walls and larger lumen than comparable arteries. Furthermore, veins have little smooth muscle tissue and are generally more pliable than arteries, wherein the variability of the diameter of certain veins can be quite large depending on the physiological conditions of a patient. In addition, most veins include valves to inhibit or prevent reverse flow, thus ensuring flow in one direction to prevent pooling.

Based upon recent studies, it is believed certain ailments may be associated with the abnormalities or improper functioning of the venous system. For example, it has been suggested Multiple Sclerosis (MS) may be caused by abnormalities in patients' cerebrospinal veins. MS is a debilitating disease in which the myelin surrounding the nerves becomes damaged, resulting in inhibition of nerve communication and impairment of physical and cognitive abilities. Abnormalities in cerebrospinal veins are believed to play a role in the pathology of certain MS patients, which can result in a resistance of venous outflow, and in turn may cause redistribution of flow to smaller, collateral veins that are unable to handle high flow. Tight endothelial junctions may then widen, and allow immune cells to pass from the circulatory system into the brain. Once these cells pass the blood-brain barrier, an autoimmune cascade can result in the demyelination and neurological symptoms of MS. This hypothesis is empirically supported by the high iron content in the brain of certain MS patients. Such levels indicate the presence of pooling of non-oxygenated blood resulting from reduced outflow of the cerebral veins.

There are several potential abnormalities that can lead to reduced cerebrospinal venous outflow. For example, MS sufferers appear to have a high prevalence of narrowing, twisting, or blockage of the veins that remove blood from the main extracranial cerebrospinal veins, such as the jugular and the azygous system. Additionally, MS sufferers also may have distended bulbous sections within their cerebrospinal veins. These bulbs can expand and cause blood accumulation and reflux as previously described. The walls of a vein are relatively weak, which may contribute to this problem with venous distension. Further, aside from anatomical abnormalities, the cytoarchitecture of the cerebral veins is such that when a person is in the supine position, the cerebrospinal veins can tend to collapse. Similarly, changes in posture, such as the hunching that can occur with age may also place compression on the cerebral veins and thereby reduce their flow. This collapse of the cerebrospinal veins may cause redirection of the blood flow.

In addition to MS, reduced cerebrospinal blood flow may be the cause of other neurologically manifesting diseases. For example, decreased blood flow to the brain in humans is associated with altered Alzheimer's Disease (AD)-related pathology. The underlying mechanisms by which hypoperfusion influences AD neuropathology remain unknown. However, the hypothesized similarities make it possible that an effective treatment for any circulatory-based causes of MS may also prove to be effective for treating AD.

A variety of treatment and devices have been developed to address certain abnormalities with the arterial system. Such treatment and devices include endoprostheses, such as stents, stent grafts, and the like. However, such implantable devices rely upon the integrity of the artery wall to support and maintain the position of the implant and therefore may not be suitable for use in the venous system, let alone to treat or address abnormalities of the venous valves.

As such, there remains a need for treatment and devices to address venous abnormalities within the venous system.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, one aspect of the disclosed subject matter is directed to an expandable intraluminal scaffold defining a longitudinal axis. The scaffold includes at least two filaments extending from a head portion disposed along the longitudinal axis at a first longitudinal end. Each of the at least two filaments includes a free end portion at a second longitudinal end opposite the head portion. The at least two filaments converge toward each other at a juncture disposed proximate the longitudinal axis between the first longitudinal end and the second longitudinal end.

The scaffold can have a variety of shapes or configurations. In general, the scaffold is free of traumatic engaging elements. The converging filaments can intersect each other at the juncture, or converge without intersection. The head portion can be a continuous contour between the at least two filaments, or can define a depression. The at least two filaments can be substantially symmetric with respect to the longitudinal axis or asymmetric with respect to the longitudinal axis, and can be spaced evenly or unevenly circumferentially with respect to the longitudinal axis. One or more of the at least two filaments can be substantially planar or can be non-planar, such as helical or spiral. The filaments can have a core-shell structure, includes one or more active agents, and/or one or more radiopaque markers or materials. The filaments can have a variety of shapes, such as a planer curve or a three-dimensional shape, and can have different cross-section shapes.

Additionally, the intraluminal scaffold can further include a constraint near the juncture to restrict the movement of the at least two filaments relative to the longitudinal axis at the juncture. The constraint can be a weld, a collar or ring, or a pivot structure. According to another aspect, the intraluminal scaffold can further include an elongated core member coupled to the scaffold. The elongated core member can include one or more ratchet features to engage the constraint, if provided.

In accordance with another aspect of the disclosed subject matter, a system is provided, which includes a delivery system having an inner member with a distal end portion and an outer sheath movable relative to the inner member. The outer sheath has a first position to cover the distal end portion of the inner member and a second position to expose the distal end portion of the inner member. The intraluminal scaffold is disposed at the distal end portion of the inner member and includes at least two filaments extending from a head portion. Each of the at least two filaments includes a free end portion at a second longitudinal end opposite the head portion. The at least two filaments converge toward each other at a juncture disposed proximate the longitudinal axis between the first longitudinal end and the second longitudinal end.

In accordance with yet another aspect of the disclosed subject matter, a method of delivering an intraluminal scaffold is provided. The method includes providing a delivery system including a delivery catheter and an intraluminal scaffold as described above, with the scaffold disposed at the distal end portion of the inner member of the catheter; positioning the distal end portion of the delivery catheter proximate a target site; and moving the outer sheath to the second position relative to the inner member to expose the scaffold at the target site. The scaffold can be deployed proximate an intraluminal valve, e.g., upstream or downstream of the valve, or can be deployed at and contact the valve. The target site can be in a vein of a patient, e.g., in an internal jugular vein.

### BRIEF DESCRIPTION OF THE DRAWINGS

FIG. 1 is a schematic side view of a representative embodiment of an intraluminal scaffold of the disclosed subject matter.

FIG. 2 is a schematic side view of another embodiment of an intraluminal scaffold of the disclosed subject matter.

FIG. 3 is a schematic side view of an intraluminal scaffold having a modified head portion.

FIG. 4 depicts an intraluminal scaffold having a free end portion having an alternative configuration.

FIG. 5A is a side view of an intraluminal scaffold including three filaments according to another aspect of the disclosed subject matter.

FIGS. 5B and 5C are cross section views taken at BC of FIG. 5A to show various cross section views of an intraluminal scaffold including three filaments.

FIG. 5D is a front view of an intraluminal scaffold of FIG. 5A.

FIGS. 6A-6B show various cross section views of an intraluminal scaffold including four filaments.

FIGS. 7A-7D are schematic side views of various intraluminal scaffolds including a plurality of filaments with different constraints.

FIG. 8 is a schematic side view of an intraluminal scaffold including an elongated core member according to another aspect of the disclosed subject matter.

FIG. 9A-9B are schematic side views of an intraluminal scaffold including an elongated core member having ratchet features according to another aspect of the disclosed subject matter.

FIGS. 10A-10C are schematic side views of intraluminal scaffolds fabricated according to the disclosed subject matter.

FIG. 11 is a cross section side view of a delivery system for deploying an intraluminal scaffold according to the disclosed subject matter.

FIGS. 12A-12C are schematic side view depicting the process for delivering an intraluminal scaffold according to the disclosed subject matter.

FIGS. 13A and 13B cross section side views depicting a delivery system having a release mechanism according to another aspect of the disclosed subject matter.

While the disclosed subject matter is capable of various modifications and alternative forms, specific embodiments thereof have been depicted in the figures, and will herein be described in detail. It should be understood, however, that the figures are not intended to limit the subject matter to the particular forms disclosed but, to the contrary, the intention is to illustrate and include all modifications, equivalents, and alternatives within the spirit and scope of the subject matter as defined by the appended claims.

### DETAILED DESCRIPTION

While the disclosed subject matter may be embodied in many different forms, reference will now be made in detail to specific embodiments of the disclosed subject, examples of which are illustrated in the accompanying drawings. This description is an exemplification of the principles of the disclosed subject and is not intended to limit the invention to the particular embodiments illustrated.

In accordance with one aspect of the disclosed matter, an intraluminal scaffold is provided which is suitable to be implanted in a body lumen, such as a blood vessel or the like, e.g., a vein, of a patient. The scaffold has a longitudinal axis, and includes at least two filaments extending from a head portion disposed along the longitudinal axis at a first longitudinal end. Each of the at least two filaments includes a free end portion at a second longitudinal end opposite the head portion. The at least two filaments converge toward each other at a juncture disposed proximate the longitudinal axis between the first longitudinal end and the second longitudinal end.

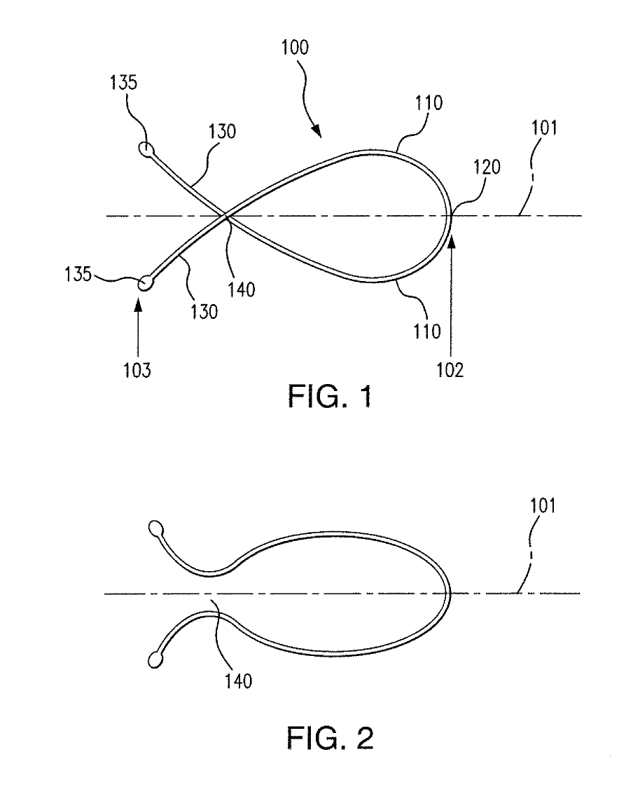

For purpose of illustration and not limitation, various embodiments of the intraluminal scaffold and related delivery system of the disclosed subject matter are described below in connection with drawings. It is noted that the figures are not to scale and certain dimensions have been exaggerated for clarity. Referring to FIG. 1, the disclosed subject matter includes an intraluminal scaffold 100 implantable in a body lumen of a patient's, such as a blood vessel, e.g., a vein, or more particularly, a cerebrospinal vein. The scaffold has a longitudinal axis 101, and includes at least two filaments 110 extending from a head portion 120 disposed along the longitudinal axis at a first longitudinal end 102. Each of the at least two filaments 110 includes a free end portion 130 at a second longitudinal end 103 that is opposite the head portion. The at least two filaments converge toward each other at a juncture 140 disposed proximate the longitudinal axis 101 between the first longitudinal end 102 and the second longitudinal end 103.

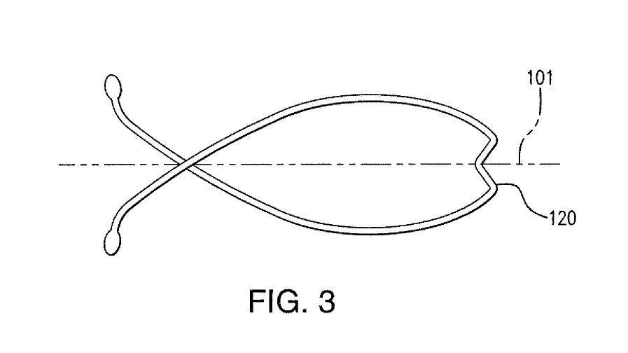

As embodied herein, the at least two filaments can converge toward each other to intersect, as depicted in FIG. 1, or the at least two filaments can converge without intersecting each other such as shown in FIG. 2. It is understood that the filaments can intersect or cross each other with or without actually contacting each other. In the three dimensional space, the filaments are not necessarily physically joined, welded, or otherwise constrained at the intersection. For example, intersecting filaments can slide along each other when being subjected to a compression load generally perpendicular to the longitudinal axis of the scaffold. The compression load can reduce the profile of the scaffold in its cross section, lengthen its longitudinal dimension, and/or alter the location of the juncture. For example, a compression load can be exerted by a confining sheath of a delivery catheter to reduce the profile of the scaffold for advancing through the vasculature of a patient. Upon release from the outer sheath, the filaments will expand outwardly from the longitudinal axis to its initial unstrained position unless or until coming into engagement with another restricting surface, such as the wall of a lumen. Either the free end portion at the second longitudinal end 103 or the body portion between the head portion and the juncture can engage the lumen wall, thereby improving the stability of the scaffold as implanted. As used herein, the juncture of converging filaments refers to a spatial zone proximate the longitudinal axis of the scaffold where the filaments converge toward each other, such as seen in a projected profile as shown in FIG. 1. Alternatively, the at least two filaments can converge toward each other, as illustrated in FIG. 2, without intersecting each other, as referenced by 140, and then extend away from each other at the free end portion 130.

The scaffold 100 as illustrated above is generally self-expanding upon release of any restraint or compressive force imposed thereon. For example, the scaffold 100 can be made of a shape memory material, or any suitable material below its yield strength, such as metals, metal alloys, polymers, and certain ceramics. The configuration as depicted in FIG. 1 is an expanded configuration. The expanded configuration can vary in profile size in response to the fluctuation of the diameter of the blood vessel in which the scaffold is implanted. Unless otherwise noted, the figures of this application schematically depict expanded configurations of the various embodiments of the flexible intraluminal scaffolds disclosed herein. It is appreciated that when implanted at a target site, e.g., in a vein, a part of the curved portion of the expanded scaffold configuration as shown in FIG. 1 can deflect to conform to the wall of the lumen.

For example, the at least two filaments of the scaffold can be sufficiently pliant to adapt to a surrounding lumen wall. Furthermore, the scaffold does not have an outward bias or hoop strength that exceeds the anticipated compressive force at the target site. The size and flexibility of the filaments can be selected such that the scaffold conforms to a body lumen, e.g., of a blood vessel such as a vein, in which the scaffold is to be implanted. By “conforming scaffold”, it is intended that the overall geometry and stiffness of the scaffold are such that the filaments can engage the lumen wall to inhibit movement within the lumen under the normal use conditions without substantially altering the diameter of the lumen at its undisturbed or natural state. However, the scaffold can be suitably sized and flexible to maintain engagement with the vessel wall in response to a change in the diameter of the vessel between its smallest diameter to its maximum anticipated diameter corresponding to different physiological states of the patient. Thus, in contrast with a supporting scaffold, such as a stent, which is configured for maintaining the patency of an artery, the conforming scaffold as disclosed herein does not urge or otherwise support the lumen wall in a predetermined diameter, but rather dynamically changes its shape to adapt to the varying size of the blood vessel at different anatomical sites and in different physiological conditions, and this allows for easy deployment, retrieval, and repositioning of the conforming scaffold within the blood vessel. If desired, the conforming scaffold can, however, have certain minimum deployed profile to prevent a total collapse of the lumen.

As such, it is not required or desired that the scaffold 100 include anchors or include elements such as the barbs or piercing elements, as commonly used in other implantable devices, to engage the lumen wall, e.g., the wall of a blood vessel. Rather, the filaments of the scaffold 100 generally have outward surfaces to atraumatically engage the wall of a blood vessel without injury to the blood vessel wall.

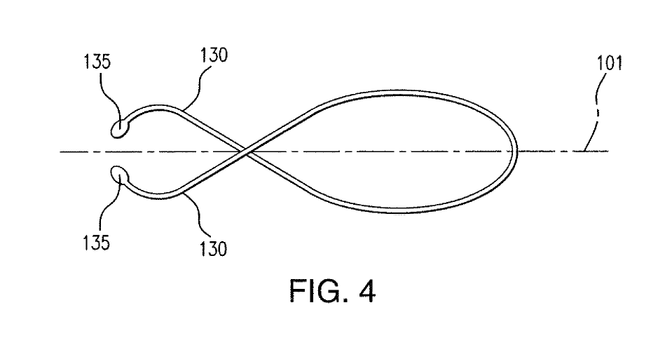

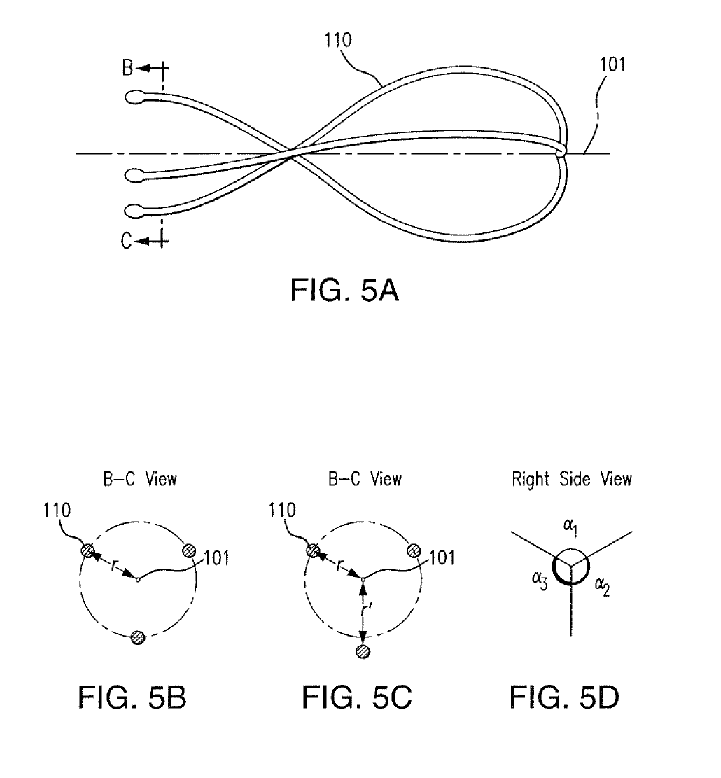

The head portion 120 of the scaffold 100 can be continuous contour between the at least two filaments 110, as depicted in FIG. 1. In such a case, the two filaments can be considered, or indeed can be formed as one continuous element. Alternatively, the head portion can be provided with a variety of configurations as desired for the intended purpose. For example, as shown in FIG. 3, the head portion can define a depression. The free end portion of the filaments can have a variety of suitable geometries. For example, as illustrated in FIG. 1, the free end portion 130 extends away from the longitudinal axis. In this manner, the free end 135 of the free end portion has the greatest distance from the longitudinal axis. Alternatively, as illustrated in FIG. 4, the free end portion can first extend away from and then inwardly towards the longitudinal axis. In this manner, the free end 135 of the free end portion is spaced more closely from the longitudinal axis than an intermediate segment of the free end portion. The free end 135 can be shaped atraumatically for contact with lumen wall, e.g., as a bulbous shape or other smooth shape as appropriate.

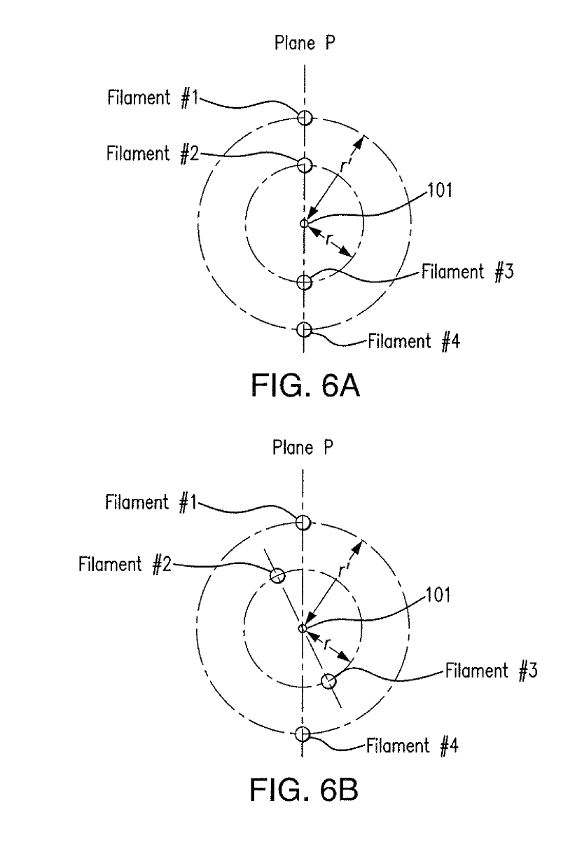

As depicted herein, the at least two filaments can be arranged substantially symmetrically with respect to the longitudinal axis. For example, and with reference to FIGS. 1-3, the two filaments can be generally mirror images of each other, and can be formed within a general plane or extend out of plane to form a three dimension curved configuration. If the scaffold includes three or more filaments, these filaments can form a generally elongated tapered cage from the head portion to the juncture. The three or more filaments can be arranged in a radially symmetric fashion with respect to the longitudinal axis. For example, as illustrated in FIG. 5A, the scaffold includes three filaments, with each of the three filaments extending radially outwardly, and spaced generally evenly about the circumference, i.e., 120 degrees. Alternatively, the filaments can be arranged asymmetrically with respect to the longitudinal axis, with the filaments having different geometric shapes and/or spaced with different diameters or angles to articulate the intended anatomy. For example, as illustrated in FIG. 5B, one of the three filaments in FIG. 5A can be spaced at a distance r′ with respect to the longitudinal axis, while the other two of the three filaments in FIG. 5A are spaced at a distance r, which is smaller than r′, with respect to the longitudinal axis. Note that in FIGS. 5B and 5C the cross section of each filaments is shown as circular; however, other shapes of the filament cross sections can be used, for example, elliptical and multilateral (or polyhedral, e.g., triangular, rectangular, etc.).

If the scaffold includes four or more filaments, the filaments can be arranged symmetrically or asymmetrically with respect to the longitudinal axis in similar fashions as illustrated in FIGS. 5A-5C, as desired. For example, for a scaffold including four filaments, each of the four filaments can extend the same distance outwardly from the longitudinal axis on an arbitrary cross section, or one or more of the four filaments can be spaced differently from the other filament(s) at an arbitrary cross section.

Circumferentially, the filaments can be arranged evenly or unevenly. The circumferential arrangement is also referred herein as the angular distribution of the filaments. For example, as shown in FIG. 5D (which is a right side view of FIG. 5A), the angles α₁, α₂, and α₃ between the three filaments can each be approximately 120 degrees, in which case, the three filaments are spaced evenly circumferentially (or form an even angular distribution). Similarly, for a scaffold including N filaments to have an even angular distribution, the angle between each pair of circumferentially neighboring filaments can be approximately 360/N degrees. Alternatively, the filaments can be arranged or spaced from each other unevenly circumferentially about the longitudinal axis, if desired or needed. For example, the angles α₁, α₂, and α₃ between the three filaments of a three-filament scaffold can be 60, 150, 150 degrees, respectively, or 90, 135, and 135 degrees, respectively. It is appreciated that an even angular distribution of filaments can allow substantially uniform expansion of the scaffold, thus provide conformability to a generally circular lumen. However, an uneven angular distribution of filaments can be desired upon the geometry the implant site of the scaffold in the blood vessel, or for influencing a structure of the blood vessel, e.g., for propping up a valve in the blood vessel, in an anisotropic fashion to achieve a desired result.

If the scaffold includes four filaments, then the four angles between each of the neighboring filaments can each be approximately 90 degrees (i.e., forming an even angular distribution). Alternatively, an uneven distribution, e.g., approximately 45, 135, 45, and 135 degrees, respectively (i.e., forming uneven angular distribution), or other angles in different circumferential arrangements, can be desired depending upon the needs and target site.

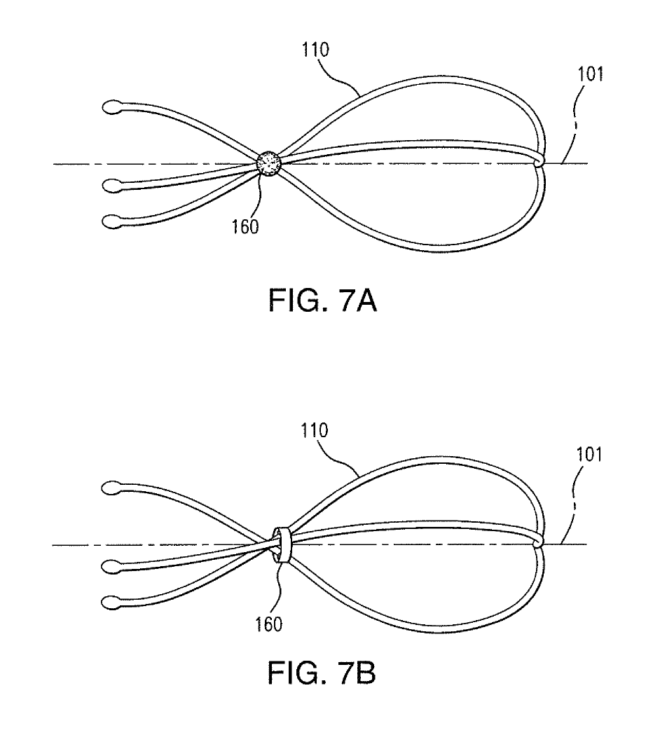

Different combinations of radial and circumferential arrangement of filaments can be selected to create a scaffold having the desired characteristics and/or properties. For example, the filaments can all be arranged in substantially a common plane with the longitudinal axis. This configuration is illustrated in FIG. 6A using a 4-filament scaffold. Two filaments (filament #2, #3) are radially symmetric to each other and spaced at a distance of r at a given cross section, and the other two filaments (filaments #1, #4) are radially symmetric to each other and spaced at a distance of r′, which is greater than r, at the same cross section. All of the four filaments lie in the same general plane P, which also encompasses the longitudinal axis 101. The angles between the filaments (#1, #2), (#2, #3), (#3, #4), and (#4, #1) are 0, 180, 0, 180, respectively. Alternatively, the filaments #2 and #3 can be slightly misaligned with filaments #1 and #4, as illustrated in FIG. 6B.

While the filaments of the scaffold as illustrated in the figures of the present application are generally depicted as simple two-dimensional curves that are co-planar with the longitudinal axis, it is understood that one or more filaments can deviate from, or include portions that deviate from the co-planar configuration, such as defining a three-dimensional curve. For example, the filaments can be helical or serpentine curves.

The material of each of the filaments can be independently selected from those materials commonly used for endoprosthesis, and can include a metal such as stainless steel, an alloy such as nitinol, a polymer, or the like. Each filament can be made of a single material, or can be multilayered, such as a core with surrounding layers of a different material. Similarly, the filaments can be of a solid construction, or include multiple finer wires braided or otherwise coupled together. Also, each of the filaments can be made of a different material and/or cross-sectional shapes and dimensions as needed or desired.

Additionally, and in accordance with another aspect of the disclosed subject matter, the filaments can be further coated with, or otherwise incorporate an active agent (for example, in reservoirs on the surface or inside of the filament), for treating, ameliorating, or inhibiting a condition of concern of a patient. For example and not limitation, the active agent can be selected from an antisense agent, an antineoplastic agent, an antiproliferative agent, an antithrombogenic agent, an anticoagulant, an antiplatelet agent, an antibiotic, an anti-inflammatory agent, a therapeutic peptide, a gene therapy agent, a cytotoxic agent, a cytostatic agent, a recombinant DNA product, a recombinant RNA product, a collagen, a collagenic derivative, a protein, a protein analog, a saccharide, a saccharide derivative, and a combination thereof.

The scaffold can be made of a radiopaque material for greater visibility during imaging, or include one or more radiopaque markers or materials. For example, one or more of the at least two filaments, or a portion thereof, can include a radiopaque marker. The radiopaque material can be a coating layer of one or more filaments, or a radiopaque marker can be attached to the surface of the filament or formed in a center bore of the filament.

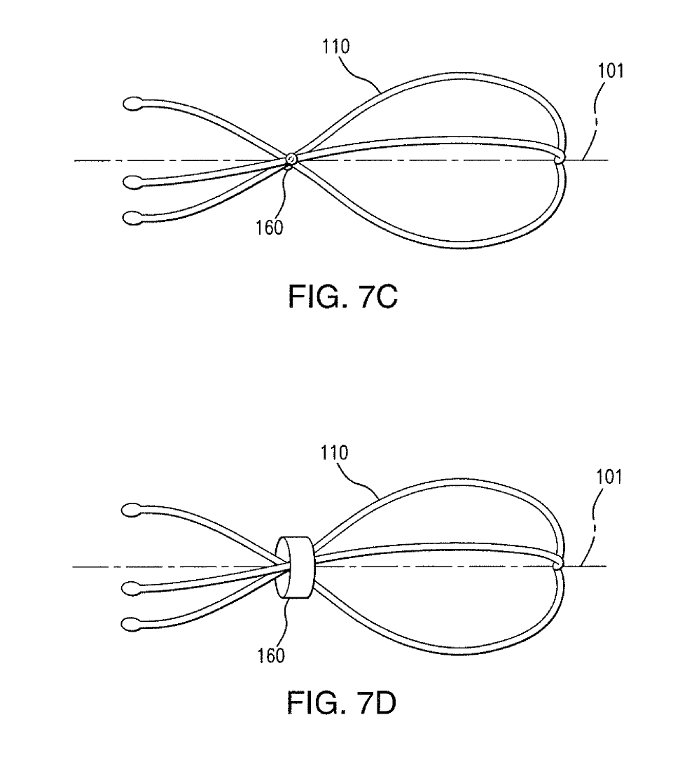

In accordance with another aspect of the disclosed subject matter, the scaffold can further include a constraint near the juncture for restricting the movement of the at least two filaments relative to the longitudinal axis near the juncture. As illustrated in FIG. 7A, the constraint 160 can be formed by directly joining the filaments together, e.g., by welding. Alternatively, the constraint 160 can be a collar coupled to the filaments, e.g., by a weld of the filaments to the collar, as illustrated in FIG. 7B. In this manner, the constraint can be formed to inhibit or prevent the transfer of stress or moment through the filament across the juncture. Alternatively, the constraint can be configured as a pivot structure, such as a rivet as illustrated in FIG. 7C, to prevent the shifting of the location of the juncture but still allow the transfer of stress or moment through the filaments. For example, if the filaments are coupled to a pivot structure at the juncture, a transverse compression load applied on one section of the scaffold can be converted to a transverse expansion force at another section of the scaffold: when a compressive load is applied at the free end portion, the body portion between the juncture and the head portion can expand transversely; when a transverse compression load is applied to the body portion between the juncture and the head portion, the free end portion can transversely expand. Alternatively, the constraint can be a free-floating ring surrounding the filaments, as illustrated in FIG. 7D. The free-floating ring engages the filaments to restrict the freedom of motion of the filaments transversely with respect to the longitudinal axis to ensure a juncture is maintained. Meanwhile, the ring still allows the filaments and the juncture to move relative to each other longitudinally.

In accordance with another aspect of the disclosed subject matter, the scaffold further includes an elongated core member coupled to the at least two filaments. Such an elongated core member can facilitate a number of functions. For example, the elongated core member can provide structural rigidity to the scaffold to facilitate deployment of the scaffold into a lumen, e.g., blood vessel. Additionally, the elongated core member can be used for providing a coupling mechanism for engagement with a delivery device, as described further below. Furthermore, the elongated core member can be used to connect one or more additional scaffolds, e.g., conforming or supporting scaffolds. The elongated core member can be made of the same material as the filaments, or of a different material, and can be any of a variety of suitable cross section shapes.

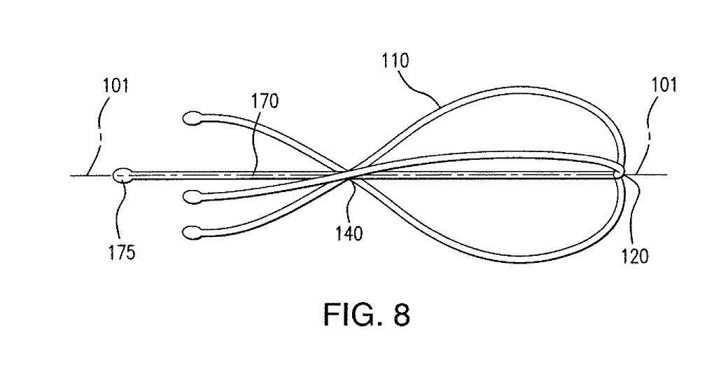

As illustrated in FIG. 8, the elongated core member 170 is coupled to the head portion 120, and substantially coincides with the longitudinal axis 101 of the scaffold so as to pass through the juncture 140. In this embodiment, the filaments can be unconstrained at the juncture and therefore freely move about the elongated core member. FIG. 8 further shows that the elongated core member extending from the head portion can have a length to extend beyond the free end portion. It is appreciated, however, that the elongated core member can have a length shorter than the longitudinal length of the filaments.

Alternatively or additionally, the elongated core member can be coupled to the filaments 120 at or proximate the juncture. If the elongated core member is coupled to both the head portion and the juncture statically, the scaffold can still be configured to collapse and expand by providing the filaments with sufficient flexibility to deflect in response to the compressive force. The elongated core member can be coupled to the filaments of the scaffold using a bonding process, e.g., thermal or chemical bonding using processes such as heat welding or adhesive gluing.

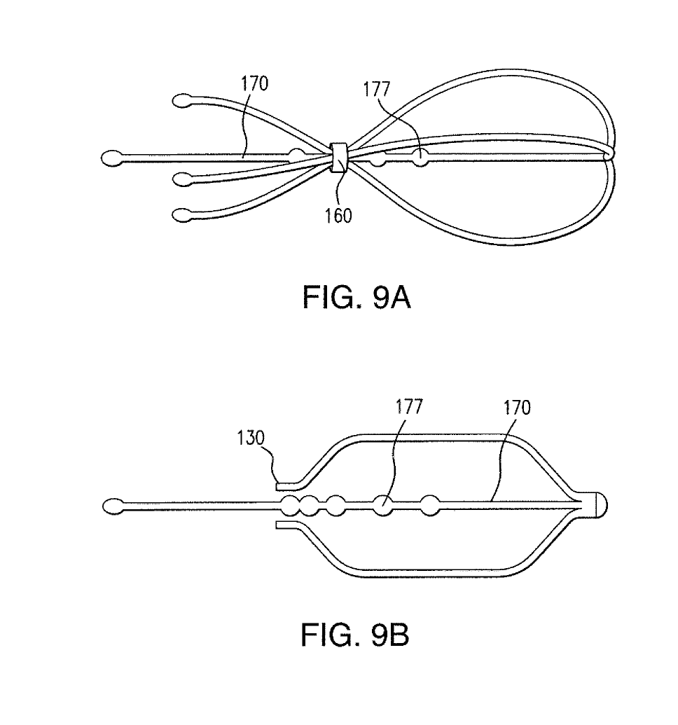

In another embodiment, the scaffold having an elongated core member can further include a constraint near the juncture, such as a constraint as described in connection with FIGS. 7B-7D to further encircle the elongated core member. The filaments can be coupled to a collar near the juncture, similar to the coupling described in connection with FIGS. 7B and 7D such that the filaments can expand and collapse uniformly and in coordination while remaining constrained radially relative to the elongated core member at the juncture, but not axially. Alternatively, the elongated core member can have one or more ratchet features 177 to cooperate with the constraint, as illustrated in FIG. 9A, to allow select discrete stable positions of the juncture relative to the elongated core member. The ratchet features can be configured to have special geometries to allow passage of the constraint in one direction, and inhibit movement in the opposite direction, as is commonly known in ratchet technology. An advantage of this embodiment is that the scaffold can be customized and sized to target location.

In the above embodiments, the collar or ring can either have a fixed diameter cross section. Further, the collar or ring can be pinched closed to inhibit relative movement, or can be provided with a certain degree of elasticity to reversibly expand and contract. Alternative to the embodiment of FIG. 9A, the free end portion of the filaments of the scaffold can terminate at the juncture, such that the free end of the filaments engage the ratchet points of the elongated core member as shown in FIG. 9B.

The elongated core member as disclosed herein can be a straight length of wire or rod, or can include one or more portions shaped as curves, such as a simple bent curve, or more complex curves such as helical, zigzag, serpentine, or the like. The elongated core member can be configured and constructed to have a sufficient rigidity to serve as a “spine” for the scaffold, e.g., to allow the scaffold to be pushed out of a delivery catheter and into a lumen. As illustrated in FIG. 8, the elongated core member 170 can include a tip 175 adapted to releasably engage a component of a delivery catheter, e.g., to an inner member of the delivery catheter. The elongated core member can include additional components, such as an element having an enlarged cross-section, an anchor, or a second scaffold of either conforming or supporting configuration.

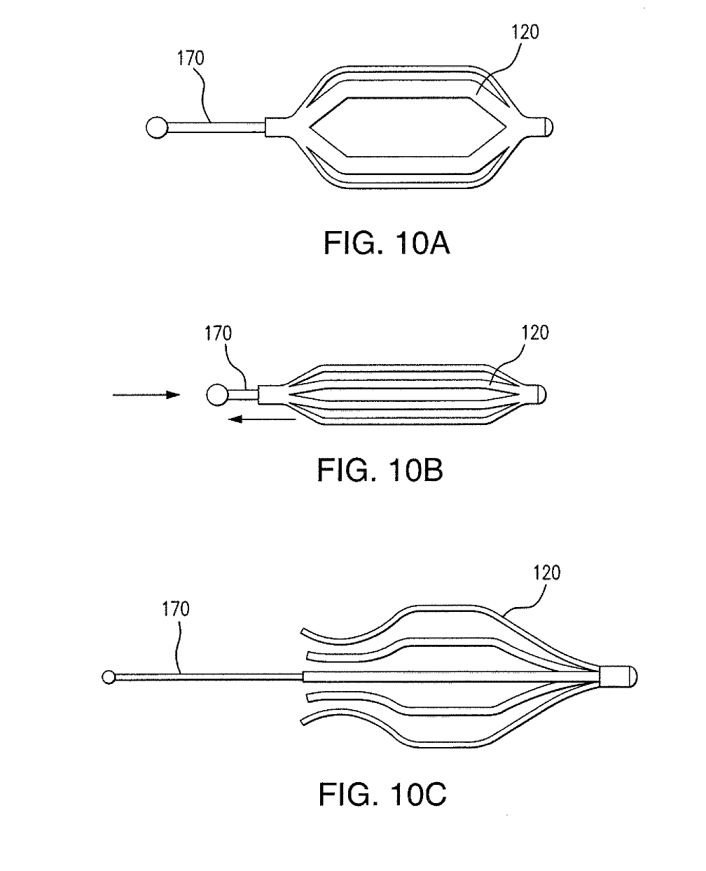

The scaffolds as described above can be fabricated as a single wire bent into suitable configuration, or by assembly and joining individual filaments together. Alternatively, the scaffold can include a hollow tube cut to define the individual filaments with suitable shape, cross section, and flexibility. For example, a hollow tube suitable for scaffold construction has an initial diameter of approximately 0.08 inches and a wall thickness of approximately 0.004 inches, although the outer diameter can be in the range of about 0.05 inches to about 0.2 inches, and the wall thickness can be in the range of about 0.002 inches to about 0.005 inches. During fabrication, cuts are be made in the tube along at least a portion of the sidewall. The cuts can be longitudinal as shown, or helical or otherwise shaped if desired. These cuts may be made using any variety of fabrication processes such as laser cutting, micromachining, abrasive cutting, or any other process known in the art. As depicted herein, the cuts are made only through a portion of the tube to allow one or both ends of the tube to remain connected. The elongated core member, if provided, can be coupled to an end of the tube. A fabricated scaffold according to the above method is illustrated in FIGS. 10A and 10B, which depict the scaffold in an expanded configuration (FIG. 10A) and a collapsed configuration (FIG. 10B). Alternatively, FIG. 10C depicts a fabricated scaffold having the cuts, and thus filaments, extend through to one end of a tube which includes a section to define a juncture of the filaments formed therefrom. The filaments are prebent or trained if made of a shape memory alloy such as nitinol to form the outwardly broad shape.

In accordance with another aspect of the disclosed subject matter, an intraluminal scaffold system is provided. The system includes a delivery system, e.g., a delivery catheter, which can be of similar construction and operations as contemplated for delivering self-expanding stents or the like. See, for example, U.S. Pat. No. 7,799,065 to Pappas, the contents of which are incorporated by reference in its entirety. For the example, the delivery catheter can include an inner member having a distal end portion and an outer sheath generally surrounding and movable relative to the inner member. The outer sheath defines a catheter lumen and has a first position to cover the distal end portion of the inner member and a second position to expose the distal end portion of the inner member, the outer sheath defining a main lumen. The system also includes an intraluminal scaffold as previously described disposed at the distal end portion of the inner member to releasably engage the distal end of the inner member of the delivery system.

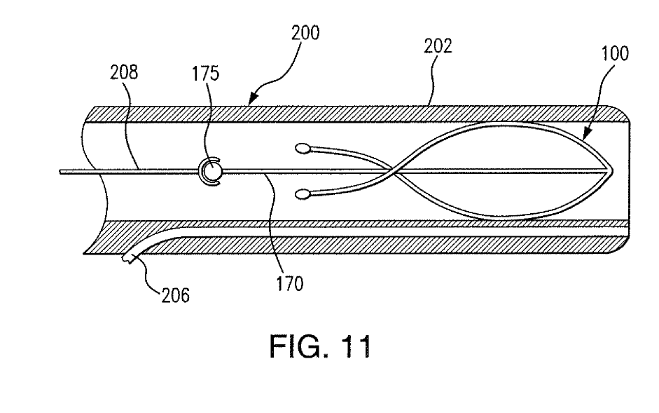

Referring to FIG. 11, the distal portion of the delivery system for deploying an intraluminal scaffold having an elongated core member is shown schematically. The delivery catheter 200 includes an outer sheath 202 to retain the scaffold 100 during delivery through a patient anatomy. The catheter is sized and configured in accordance with the intended use and target site. For example, the outer diameter of the catheter can be between about 4 Fr to 6 Fr in diameter.

The outer sheath is made of any suitable material as known in the art, including single layer or multi-layer construction, and sized and configured to constrain the scaffold in a low profile condition. Additionally or alternatively, the scaffold can further include a pullwire wound on the filaments to reduce the profile of the scaffold before deployment, which can be removed after the scaffold is exposed outside of the delivery system. For purpose of illustration and not limitation, FIG. 11 shows a scaffold in a low profile condition, although it will be understood the degree of compression can be greater to reduce the profile of the scaffold during delivery. As depicted herein, the scaffold includes an elongated core member 170 having a tip 175 to releasably engages the inner member 208, also referred herein as a pusher, of the catheter. Generally, the inner member 208 has a distal end configured to engage or mate with the tip of the scaffold in a stable manner. For example, but not limitation, FIG. 11 depicts the distal end portion of the inner member with a cup geometry. Alternatively, the distal portion of the inner member can have a tube with a back support geometry, or any other geometry to engage the tip of the elongated core member of the scaffold. As an alternative, the inner member can engage the juncture, or the free end portion of one or more filaments of the scaffold if no core member is provided.

The inner member is generally configured for longitudinal strength but axial flexibility. For example, the inner member can be constructed from a metallic wire that extends the length of the catheter. The outer sheath is movable relative to the inner member to expose the scaffold at the distal portion of the inner member. Actuation can occur by manually pushing the inner member, or by retracting the outer sheath by conventional means of actuation such as the rotation of a knob or gear that engages the pusher wire, as known in the art of stent delivery. Various other actuation mechanisms and catheter features consistent with the disclosed subject matter can be provided. For example, the delivery catheter can be either configured for over the wire (OTW) or rapid exchange (RX) guidewire deployment. Thus, in one embodiment, the catheter includes an RX guidewire lumen 206, as shown in FIG. 11. The guidewire lumen has a diameter that is suitable for the passage of a guidewire with an average diameter of between about 0.014 inches to about 0.035 inches. As shown, the guidewire lumen may run from a distal end of the delivery system to an exit port along the side of the catheter.

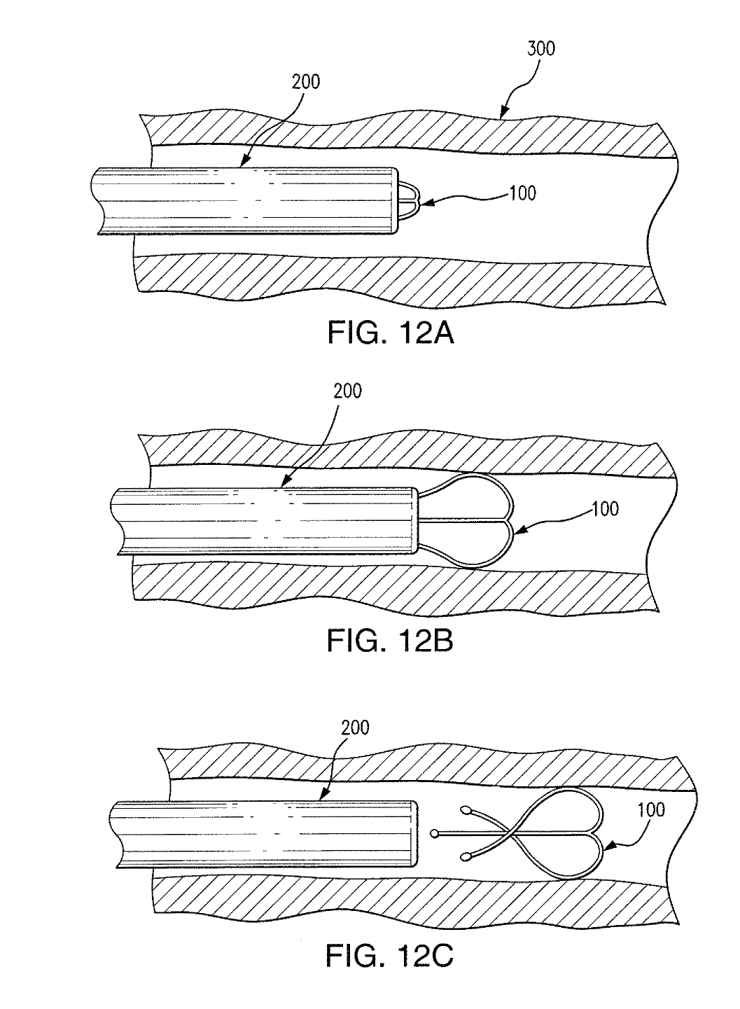

In accordance with another aspect of the disclosed subject matter, a method of delivering an intraluminal scaffold is provided. The method includes providing a delivery system including a delivery catheter and an intraluminal scaffold as described above, with the scaffold disposed at the distal end portion of the inner member of the catheter. The distal end portion of the delivery catheter is positioned proximate a target site. The outer sheath is moved to the second position relative to the inner member to expose the scaffold at the target site. The method is illustrated schematically in FIGS. 12A-12C. The delivery system as described above is advanced to a target site, such as a site in a blood vessel 300. The scaffold is deployed from the delivery catheter by moving the outer sheath relative to the inner member, e.g., by advancing the inner member or by retracting the outer sheath of the catheter. It is noted in FIG. 12A that the guidewire has been retracted within the delivery catheter to allow for scaffold deployment without the risk of entanglement of the guidewire. Alternatively, the guidewire can remain in place and retracted from between the scaffold and the vessel wall at a later point. As shown in FIG. 12B, the scaffold is exposed beyond the distal end of the outer sheath until it contacts the vessel wall of the vasculature. It will be appreciated that the scaffold may contact the vessel at a more proximal location than that shown depending upon the configuration of the scaffold. Finally, as shown in FIG. 12C, the scaffold is completely deployed from the deliver catheter and is positioned within the vessel. The delivery system can then be retracted from the anatomy.

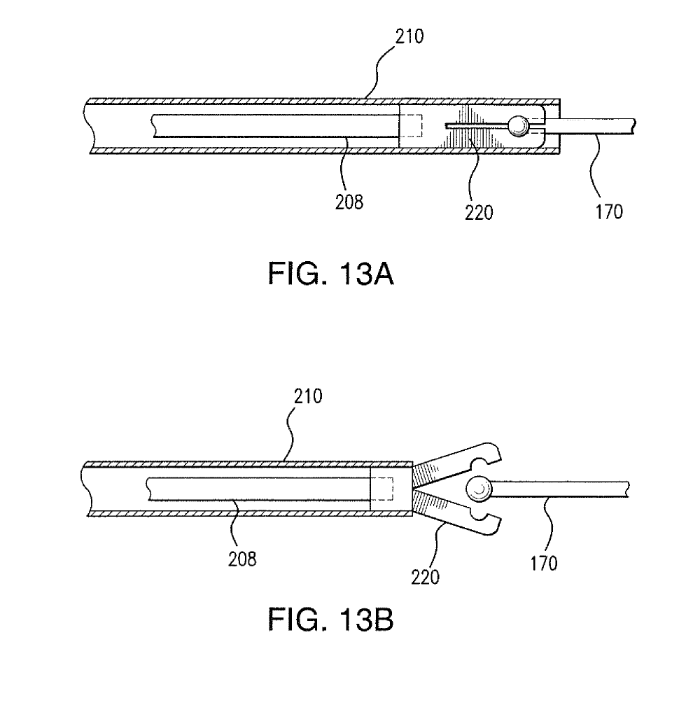

In accordance with another aspect of the disclosed subject matter, the distal end of the inner member of the delivery catheter can include a jaw mechanism to capture a portion of the scaffold, such as the tip of the elongated core member of the scaffold, when the scaffold is constrained by the outer sheath of the catheter. The jaw is configured to open when exposed outside of the outer sheath, and thus release the scaffold when fully deployed. An embodiment according to this aspect is illustrated in FIGS. 13A and 13B. While in a forward position, the outer catheter sheath 210 surrounds the inner member 208, which includes a jaw mechanism 220 at its distal end, as illustrated in FIG. 13A. In this position, the catheter sheath 210 constrains the jaw mechanism 220 in a first, low profile. When the catheter sheath 210 is retracted relative to the pusher, the jaw expands to a second, large profile, as shown in FIG. 13B. Similarly, the jaw can be used to engage and retrieve the scaffold during or after deployment. That is, the jaw can be positioned in proximity of the tip of the elongated core member and the outer sheath can then be moved distally toward the extended position to collapse the jaw over the tip of the elongated core member of the scaffold. Further extension of the outer sheath can cause the scaffold to be fully received within the sheath. In this manner, the scaffold can be retrieved and removed, or redeployed in a new position.

The jaw mechanism can be formed of a single piece construction of shape memory metals through heat setting, or otherwise to bias toward the open profile. The jaw can be formed, for example, by laser cutting a portion of a tube or rod in a lengthwise direction. This forms an alligator jaw-like geometry. To engage an end feature of the scaffold, such as a knob, the jaw can further be formed to include a mating geometry. This can be accomplished in a number of ways. For example, the internal surface of the jaw can be micro-machined. Alternatively, in the case of a tube, the tube can be swaged at two axially offset locations in order to form a groove to receive the knob.

In the above method, the target site in which the scaffold is to be implanted can be proximate to a valve in a body lumen, such as a blood vessel. In one embodiment, the scaffold can be deployed upstream of the valve in the blood vessel. In another embodiment, the scaffold can be deployed downstream of the valve in the blood vessel. In yet another embodiment, scaffold can be deployed such that the scaffold overlaps and directly engages the valve. As previously noted, the scaffold system and method are particularly suited for the venous system, and especially the internal jugular vein, although other indications and target sites are contemplated.

While illustrative embodiments of the invention have been disclosed herein, numerous modifications and other embodiments may be devised by those skilled in the art in accordance with the invention. For example, the various features depicted and described in the embodiments herein can be altered or combined to obtain desired scaffold characteristics in accordance with the invention. Therefore, it will be understood that the appended claims are intended to include such modifications and embodiments, which are within the spirit and scope of the present invention.

1. An expandable intraluminal scaffold defining a longitudinal axis, the scaffold comprising:
at least two filaments extending from a head portion disposed along the longitudinal axis at a first longitudinal end, each of the at least two filaments including a free end portion at a second longitudinal end opposite the head portion, the at least two filaments converge toward each other at a juncture disposed proximate the longitudinal axis between the first longitudinal end and the second longitudinal end.

2. The expandable intraluminal scaffold of claim 1, wherein the at least two filaments of the scaffold are sufficiently pliant to adapt to a surrounding lumen wall.

3. The expandable intraluminal scaffold of claim 1, wherein the at least two filaments intersect each other at the juncture.

4. The expandable intraluminal scaffold of claim 1, wherein the scaffold is free of traumatic engaging elements.

5. The expandable intraluminal scaffold of claim 1, wherein the head portion is a continuous contour between the at least two filaments.

6. The expandable intraluminal scaffold of claim 1, wherein the free end portion of each element extends away from the longitudinal axis with the free end portion having a free end disposed from the longitudinal axis.

7. The expandable intraluminal scaffold of claim 1, wherein the at least two filaments include at least three filaments.

8. The expandable intraluminal scaffold of claim 7, wherein the at least three filaments form a generally elongated tapered cage.

9. The expandable intraluminal scaffold of claim 7, wherein the at least three filaments are spaced at different distances from the longitudinal axis at a cross section perpendicular to the longitudinal axis.

10. The expandable intraluminal scaffold of claim 7, wherein the at least three filaments are substantially co-planar.

11. The expandable intraluminal scaffold of claim 1, wherein at least one of the at least two filaments is non-planar.

12. The expandable intraluminal scaffold of claim 1, wherein at least one of the at least two filaments includes an active agent.

13. The expandable intraluminal scaffold of claim 1, wherein at least one of the at least two filaments includes a radiopaque marker.

14. The expandable intraluminal scaffold of claim 1, further including a constraint near the juncture to restrict the movement of the at least two filaments relative to the longitudinal axis at the juncture.

15. The expandable intraluminal scaffold of claim 1, further including an elongated core member coupled to the at least two filaments.

16. The expandable intraluminal scaffold of claim 15, wherein the elongated core member is coupled to the at least two filaments proximate the head portion.

17. The expandable intraluminal scaffold of claim 15, wherein the elongated core member is coupled to the at least two filaments proximate the juncture.

18. The expandable intraluminal scaffold of claim 17, wherein the elongated core member is coupled to the at least two filaments by a constraint, and wherein the elongated core member includes one or more ratchet features to engage the constraint.

19. The expandable intraluminal scaffold of claim 15, wherein the elongated core member has a tip adapted to releasably engage a delivery system.

20. The expandable intraluminal scaffold of claim 1, further comprising a pullwire wound on the at least two filaments.

21. A system, comprising:
a delivery system having an inner member having a distal end portion and an outer sheath generally surrounding and movable relative to the inner member, the outer sheath defining a catheter lumen and having a first position to cover the distal end portion of the inner member and a second position to expose the distal end portion of the inner member;
an intraluminal scaffold comprising at least two filaments extending from a head portion disposed along the longitudinal axis at a first longitudinal end, each of the at least two filaments including a free end portion at a second longitudinal end opposite the head portion, the at least two filaments converge toward each other at a juncture disposed proximate the longitudinal axis between the first longitudinal end and the second longitudinal end;
wherein the intraluminal scaffold releasably engage the distal end of the inner member of the delivery system.

22. The system of claim 21, wherein the intraluminal scaffold further comprises an elongated core member having a tip releasably engaged with the inner member.

23. The system of claim 22, wherein the distal end of the inner member includes a jaw mechanism to capture the tip of the elongated core member of the intraluminal scaffold when the outer sheath is in the first position and to release the tip when the outer sheath is in the second position.

24. A method for delivering an intraluminal scaffold, comprising
providing a system comprising:
a delivery system having an inner member having a distal end portion and an outer sheath movable relative to the inner member, the outer sheath having a first position to cover the distal end portion of the inner member and a second position to expose the distal end portion of the inner member;
an intraluminal scaffold comprising at least two filaments extending from a head portion disposed along the longitudinal axis at a first longitudinal end, each of the at least two filaments including a free end portion at a second longitudinal end opposite the head portion, the at least two filaments converge toward each other at a juncture disposed proximate the longitudinal axis between the first longitudinal end and the second longitudinal end, the intraluminal scaffold being disposed at the distal end portion of the inner member,
positioning the delivery system with the distal end portion disposed proximate a target site in an intraluminal system of a patient;
moving the outer sheath to the second position relative to the inner member to expose the scaffold at the target site.

25. The method of claim 24, wherein the target site is proximate a valve.

26. The method of claim 24, wherein the target site is one of upstream of the valve, downstream of the valve, or the portion of the lumen containing the valve.
